SEQUENCE LISTING (1 sequence)

SEQ ID NO: 1 - protein, 2413 aa, Gallus gallus
GAGTGGCGCAGCGTAGAAAGCGAGACGGATCGAGAAAAAACAGGAAGAAGCCCGATCTGGCTGCAATCTACGGGAGAGGGTTGGGCTAGAGAGTGGGCGCTACGCTTCTGATTGGGCAGGAGGCAAGGGGCGGGCGCTCTTCGGCTAGTCCGGGAGGCGGATTCGGTCAACTGCGGCAGTCGGGTGTCTGGATTGGTCCTTAGCGTTCTGGCAGGTTCCAGAAGAAGGCTAAGCGGACTATAAAGAGGGCGCGACGGCCGTAACGGCAGATCGCGCCGCAGACAGCAGCGAGAAGCGGGCGGAGGAGACGTGACTGCGAGCGAGCAAGTGACTGGCGGAGCGAGTGGCTGACTGACCAAGAGGAATCTATCATCATGTCTGGCAAAGGGCCGGCCATCGGCATCGATCTGGGCACCACGTATTCTTGCGTGGGTGTCTTCCAGCATGGCAAAGTGGAGATCATTGCCAACGACCAGGGGAACCGCACCACACCCAGCTATGTGGCCTTCACCGATACAGAGCGCCTCATCGGGGATGCTGCCAAGAACCAAGTGGCAATGAACCCCACCAACACCATCTTTGATGCCAAGCGTCTCATCGGCCGCAAGTATGATGACCCCACAGTGCAGTCGGACATGAAGCACTGGCCGTTCCGTGTGGTGAACGAGGGTGGCAAGCCCAAGGTGCAGGTGGAGTACAAGGGTGAGATGAAGACCTTCTTCCCAGAGGAGATCAGCTCTATGGTGCTCACCAAGATGAAGGAGATTGCTGAGGCCTATCTGGGAAAAAAGGTAGAGACTGCTGTTATCACAGTGCCCGCTTACTTCAACGACTCCCAGCGCCAGGCCACCAAAGATGCTGGCACCATCACTGGGCTTAACGTGATGCGTATTATCAATGAGCCCACAGCAGCTGCTATTGCCTATGGCTTGGATAAGAAAGGTACCCGGGCTGGAGAGAAGAATGTGCTCATCTTTGACTTGGGAGGGGGCACTTTTGATGTGTCCATCCTTACCATTGAGGATGGCATCTTTGAGGTGAAGTCCACAGCTGGGGACACCCACCTAGGTGGGGAGGACTTTGACAACCGCATGGTAAACCGTTTTGTAGAAGAGTTCAAGGGTAAGCACAAGCGTGACAATGCTGGCAATAAGCGAGCAGTGAGGCGTCTGCGTACAGCTTGTGAGAGGGCGAGGCGTACTCTGAGCTCTTCCACGCAAGCCAGCATTGAGATTGACTCCCTCTTTGAGGGCATTGACTTCTACACCTCCATCACTCGTGCCCGCTTTGAGGAACTCAATGCTGATCTTTTCCGTGGTACCCTGGAGCCAGTGGAGAAGGCCCTGCGTGATGCCAAGCTTGATAAGGGCCAGATCCAGGAGATTGTGCTTGTGGGGGGCTCCACTCGTATTCCTAAGATCCAGAAGTTGCTGCAAGATTTCTTCAATGGCAAAGAGCTGAACAAGAGCATCAATCCAGATGAAGCTGTTGCTTATGGTGCCGCTGTGCAAGCAGCTATCCTCATGGGAGACAAGTCTGAAAATGTGCAAGATCTGCTCCTGTTGGATGTCACCCCCCTGTCCCTGGGCATCGAGACAGCTGGTGGAGTGATGACTGCTCTCATCAAGCGTAACACCACCATTCCCACCAAACAAACACAGACCTTCACCACCTACTCAGACAACCAGAGCAGTGTCCTCGTCCAGGTGTATGAAGGTGAGAGGGCTATGACAAAGGACAACAACTTGCTGGGCAAGTTTGACCTAACAGGCATCCCCCCGGCACCCCGTGGAGTTCCTCAGATCGAGGTCACTTTTGACATAGATGCTAATGGTATCCTGAACGTCAGTGCTGTGGACAAGAGTACAGGGAAGGAGAACAAGATAACCATCACCAATGACAAGGGTCGCCTTAGCAAAGATGATATTGACCGTATGGTACAAGAAGCAGAGAAATACAAAGCAGAGGATGAAGCCAACAGAGATAGGGTGGGAGCCAAGAACTCCCTTGAGTCGTATACTTACAACATGAAGCAGACAGTGGAGGATGAGAAACTGAAGGGAAAGATCAGTGACCAGGACAAGCAGAAAGTGCTCGACAAGTGCCAGGAGGTGATCAGTTCGCTTGACCGAAACCAGATGGCAGAGAAAGAAGAGTATGAGCACAAGCAGAAAGAGCTGGAGAAACTCTGCAACCCGATTGTCACAAAACTGTACCAGGGAGCTGGAGGAGCTGGGGCAGGTGGCTCCGGTGGCCCAACCATTGAAGAAGTAGATTAAAAAGACTCTTAAACTATAGACTGGTTTATGGACAGTCACTCCATTCTTTGCTTTATATTTTTTTCTAACGTTTAAGGAAAAACGTCATTGCCAATAACAGAGTTTATTCTGTTGGGTGTGTATAAAGGCAAATC

(19) United States
(12) Patent Application Publication
(10) Pub. No.: US 2019/0072558 A1
(43) Pub. Date: Mar. 7, 2019

(54) METHOD OF DETECTING AND DETERMINING STRESS IN POULTRY BASED ON EXPRESSION OF HSP70 IN GROWING FEATHERS

(71) Applicant: BOARD OF TRUSTEES OF THE UNIVERSITY OF ARKANSAS, LITTLE ROCK, AR (US)

(72) Inventor: SAMI DRIDI, Fayetteville, AR (US)

(73) Assignee: BOARD OF TRUSTEES OF THE UNIVERSITY OF ARKANSAS, LITTLE ROCK, AR (US)

(21) Appl. No.: 16/059,191

(22) Filed: Aug. 9, 2018

Related U.S. Application Data
(60) Provisional application No. 62/549,751, filed on Aug. 24, 2017.

(51) Int. Cl. G01N 33/574; A01K 29/00

(52) U.S. Cl. CPC G01N 33/57484; A01K 2227/30; A01K 29/005

(57) ABSTRACT

This invention relates generally to a method of detecting and determining stress in poultry based on expression of HSP70 in growing feathers. More particularly, the invention relates to a non-invasive and accurate process to evaluate stress levels, such as from environmental heat or cold, transport, and/or feed restriction, in poultry species, such as broiler chickens, layers, turkeys, breeders, and/or quail. The method uses feather HSP70 expression as a non-invasive marker for stress in poultry species, and also provides for a method to rapidly evaluate and continuously monitor dynamic change and time-course stress induced by stress load in the same individual poultry.

### CROSS REFERENCE TO RELATED APPLICATIONS

This application claims the benefit of U.S. Provisional Patent Application Ser. No. 62/549,751 filed on Aug. 24, 2017, and incorporates said provisional application by reference into this document as if fully set out at this point.

### BACKGROUND OF THE INVENTION

### 1. Field of the Invention

This invention relates generally to a method of detecting and determining stress in poultry based on expression of HSP70 in growing feathers, and in particular to a method of using HSP70 gene and protein expression in growing feathers as a non-invasive marker for stress in poultry.

### 2. Description of the Related Art

Animal agriculture and particularly poultry is facing substantial challenges from a steep projected increase in demand for high quality animal protein and the need to adapt to higher temperatures due to climate change. Large, abrupt, and widespread extreme heat waves have occurred repeatedly in the past and are predicted to increase for the next century.

Environmental heat stress (HS) impacts every aspect of animal lives and their very existence. It can result in heat-related discomfort, illness, multiple organ damage, and in extreme cases can lead to death. Poultry do not have sweat glands and do not cope well with high environmental temperature. In broiler chickens, which play a key role in the U.S. and worldwide meat production, there is a strong negative effect of HS on well-being, growth, feed efficiency, meat yield and mortality. Such effects will take a heavy toll during the next decades as the distribution of heat anomalies continues to increase. The U.S. poultry industries lose over $350 million annually, and global livestock loses are in the billions.

Cells respond to elevated temperature by activating heat shock gene transcription and the rapid synthesis of heat shock proteins. Heat shock proteins are often the prominent proteins to be expressed following environmental insults. They are molecular chaperones, involved in many cellular functions such as protein folding/unfolding, assembling/disassembling, transport, maturation and degradation. They play a key role in cellular defense, repair, and detoxification. Their expression has been shown to be induced in many cells and tissues (liver, muscle, heart) from many species including chicken following heat stress.

The stress levels in chickens is generally determined by the measurement of circulating corticosterone concentrations. Most strategies used to evaluate corticosterone concentrations are invasive based on blood sampling, which by itself, is stressful for the chickens, or internal tissues collection after animal euthanasia. Additionally, manipulation of chickens may induce variations in corticosterone levels and may lead to misinterpretations.

Thus, there is a critical need for identification of a non-invasive and accurate process to evaluate stress levels in chickens.

It is therefore desirable to provide a method of non-invasively detecting and determining stress in poultry based on expression of hsp70 in growing feathers.

It is further desirable to provide a method of using HSP70 gene and protein expression in growing feathers as a non-invasive marker for stress in poultry.

It is still further desirable to provide a method to rapidly evaluate and continuously monitor dynamic change and time-course stress induced by stress load in the same individual poultry.

Before proceeding to a detailed description of the invention, however, it should be noted and remembered that the description of the invention which follows, together with the accompanying drawings, should not be construed as limiting the invention to the examples (or embodiments) shown and described. This is so because those skilled in the art to which the invention pertains will be able to devise other forms of this invention within the ambit of the appended claims.

### SUMMARY OF THE INVENTION

In general, in a first aspect, the invention relates to a non-invasive method of detecting stress in a poultry species. The method includes obtaining a growing feather sample from the poultry species, and then detecting the expression of HSP70 in the growing feather sample, whereby the expression of HSP70 is indicative of the stress in the poultry species. The sample can be non-invasively obtained without biopsy or euthanasia. In accordance with this aspect of the invention, the method can also include assaying the growing feather sample for the expression level of at least one stress-related marker, with the marker being a protein encoded by the gene of HSP70 or fragments of the protein which are immunoreacitve to HSP70. In addition, the method can include the step of determining the amount of HSP70 in the growing feather sample, where an elevated amount of HSP70 indicates the presence of stress in the poultry species. Lastly, the method may include diagnosing the poultry species with stress when HSP70 is detected in the sample isolated from the feather sample.

In general, in a second aspect, the invention relates to a method of detecting stress in a poultry species. In accordance with this aspect of the invention, the method includes non-invasively obtaining, without biopsy or euthanasia, a sample of at least one growing feather from the poultry species, and then assaying the growing feather sample for the expression level of at least one stress-related marker. The marker is a protein encoded by the gene of HSP70 or fragments of the protein which are immunoreacitve to HSP70, and the expression of HSP70 is indicative of the stress in the poultry species.

In general, in a third aspect, the invention relates to a method of determining stress induced by stress in a living poultry subject. According to this aspect of the invention, the method includes obtaining a biological sample of at least one growing feather from the living poultry subject; extracting or otherwise isolating genomic DNA from the biological sample; treating the genomic DNA, or a fragment thereof, with one or more reagents specific for HSP70 as a stress-related marker; detecting the expression HSP70; and determining the stress in the living poultry subject when the presence of HSP70 from the biological sample is detected. The method can also include the step of determining the amount of HSP70 in the biological sample, where an elevated amount of HSP70 indicates the presence of stress in the living poultry subject.

The stress of the poultry species or subject can be stress induced by environmental heat or cold, feed restrictions, transport or a combination thereof. The poultry species or subjects can be chickens, turkeys, or quail, or more particularly broiler chickens.

In general, in a fourth aspect, the invention relates to a polymerase chain reaction (PCR) feather-HSP70 kit or an enzyme-linked immunosorbent assay (ELISA) feather-HSP70 kit for use with the method of detecting and determining stress in a poultry species disclosed herein.

The foregoing has outlined in broad terms some of the more important features of the invention disclosed herein so that the detailed description that follows may be more clearly understood, and so that the contribution of the inventors to the art may be better appreciated. The invention is not to be limited in its application to the details of the construction and to the arrangements of the components set forth in the following description or illustrated in the drawings. Rather, the invention is capable of other embodiments and of being practiced and carried out in various other ways not specifically enumerated herein. Finally, it should be understood that the phraseology and terminology employed herein are for the purpose of description and should not be regarded as limiting, unless the specification specifically so limits the invention.

### BRIEF DESCRIPTION OF THE DRAWINGS

These and further aspects of the invention are described in detail in the following examples and accompanying drawings.

FIG. 1 graphically illustrates data uploaded from HOBO loggers showing that the ambient temperature in the barn increased progressively during the day to reach 29-30° C. early in the afternoon compared to 23-24° C. in the morning.

FIG. 2A graphically illustrates heat stress increased core body temperature in broilers as measured using Thermochron® loggers.

FIG. 2B is a bar graph illustrating heat stress increased core body temperature in broilers as measured using Thermochron® loggers.

FIG. 2C is a thermal image showing heat stress increased skin and surface body temperature in broilers.

FIG. 2D is another thermal image showing heat stress increased skin and surface body temperature in broilers.

FIG. 3A is a Western blot depicting HSP70 mRNA and protein highly expressed in chicken feathers in accordance with an illustrative embodiment of the invention disclosed herein.

FIG. 3B is a Western blot depicting HSP70 mRNA and protein highly expressed in chicken feathers in accordance with an illustrative embodiment of the invention disclosed herein.

FIG. 3C graphically illustrates HSP70 mRNA and HSP70/β-actin mRNA are induced by heat stress in accordance with an illustrative embodiment of the invention disclosed herein.

FIG. 3D is a Western blot depicting HSP70 mRNA and protein highly expressed in chicken feathers in accordance with an illustrative embodiment of the invention disclosed herein.

FIG. 3E graphically illustrates HSP70 mRNA and HSP70/β-actin mRNA are induced by heat stress in accordance with an illustrative embodiment of the invention disclosed herein.

FIG. 3F is a Western blot depicting HSP70 mRNA and protein highly expressed in chicken feathers in accordance with an illustrative embodiment of the invention disclosed herein.

FIG. 3G graphically illustrates HSP70 mRNA and HSP70/β-actin mRNA are induced by heat stress in accordance with an illustrative embodiment of the invention disclosed herein.

FIG. 4 is a photograph of a location of growing feathers on a broiler chicken.

### DETAILED DESCRIPTION OF THE INVENTION

While this invention is susceptible of embodiment in many different forms, there is shown in the drawings, and will herein be described hereinafter in detail, some specific embodiments of the invention. It should be understood, however, that the present disclosure is to be considered an exemplification of the principles of the invention and is not intended to limit the invention to the specific embodiments so described.

This invention relates generally to a method of detecting and determining stress in poultry based on expression of HSP70 in growing feathers. More particularly, the invention relates to a non-invasive and accurate process to evaluate stress levels, such as from environmental heat or cold, transport, and/or feed restriction, in poultry species, such as broiler chickens, layers, turkeys, breeders, and/or quail. The method uses feather HSP70 expression as a non-invasive marker for stress in poultry species, and also provides for a method to rapidly evaluate and continuously monitor dynamic change and time-course stress induced by stress load in the same individual poultry. As the growing feathers regenerate, the method can measure the time-course and dynamic change of stress levels in the same individual without biopsy or animal euthanasia.

The inventive method includes non-invasively obtaining a growing feather sample from a living poultry species or subject without biopsy or euthanasia. A polymerase chain reaction kit, an enzyme-linked immunosorbent assay (ELISA) kit or the like is then used to detect the presence of HSP70 in the growing feather sample. The growing feather sample is assayed for the expression level of at least one stress-related marker, with the marker being a protein encoded by the gene of HSP70 or fragments of the protein which are immunoreacitve to HSP70. In addition, the amount of HSP70 expressed in the growing feather sample can be determined, and an elevated amount of HSP70 indicates the presence of stress in the poultry species. Lastly, the method may include diagnosing the poultry species with stress when HSP70 is detected in the sample isolated from the feather sample.

In addition, the invention can include an ELISA or a PCR feather-HSP70 kit configured for detecting and determining stress in poultry based on expression of HSP70 in growing feathers.

### Examples

The method of detecting and determining stress in poultry based on expression of HSP70 in growing feathers disclosed herein is further illustrated by the following examples, which are provided for the purpose of demonstration rather than limitation.

Animals:

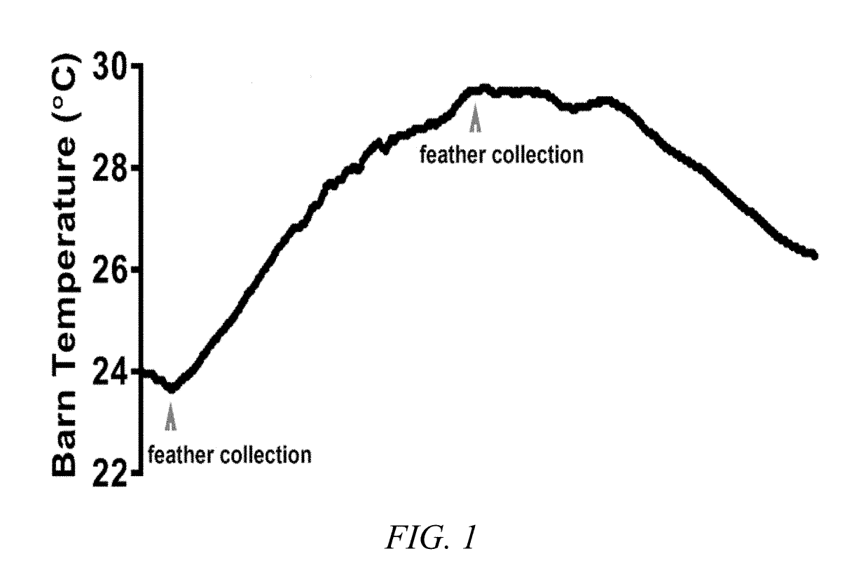

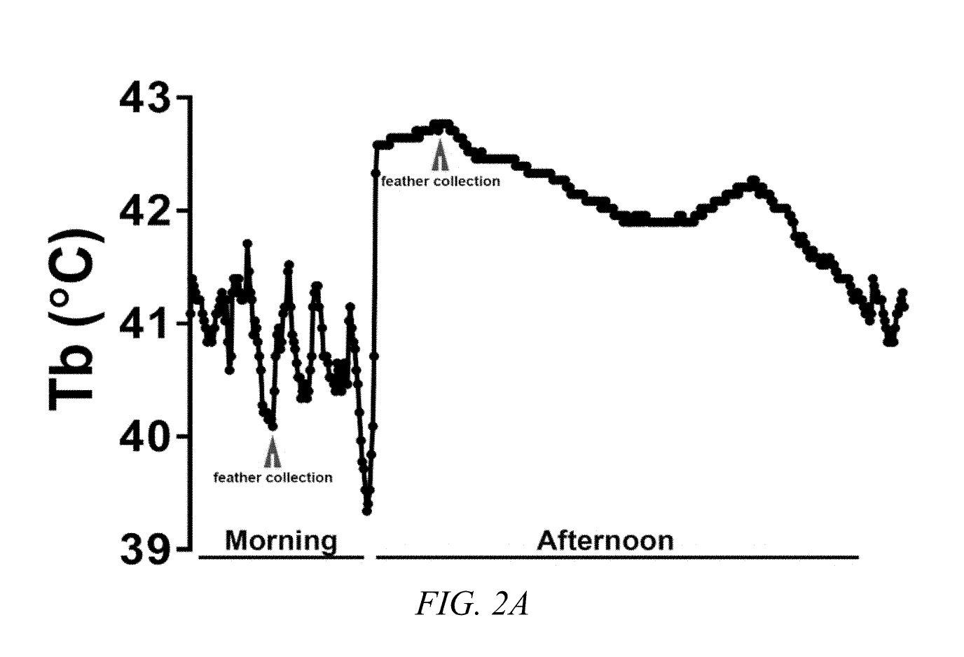

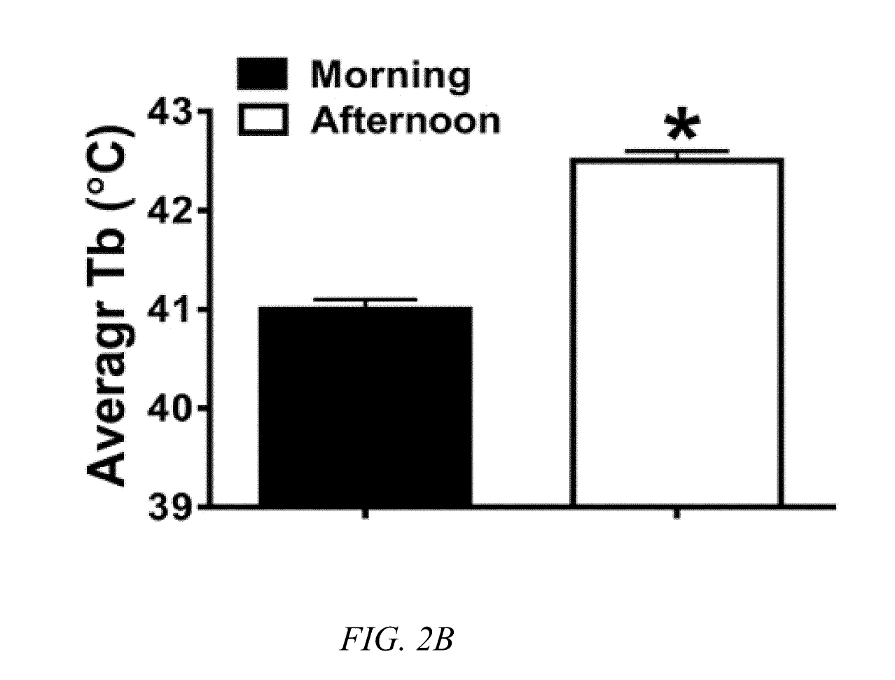

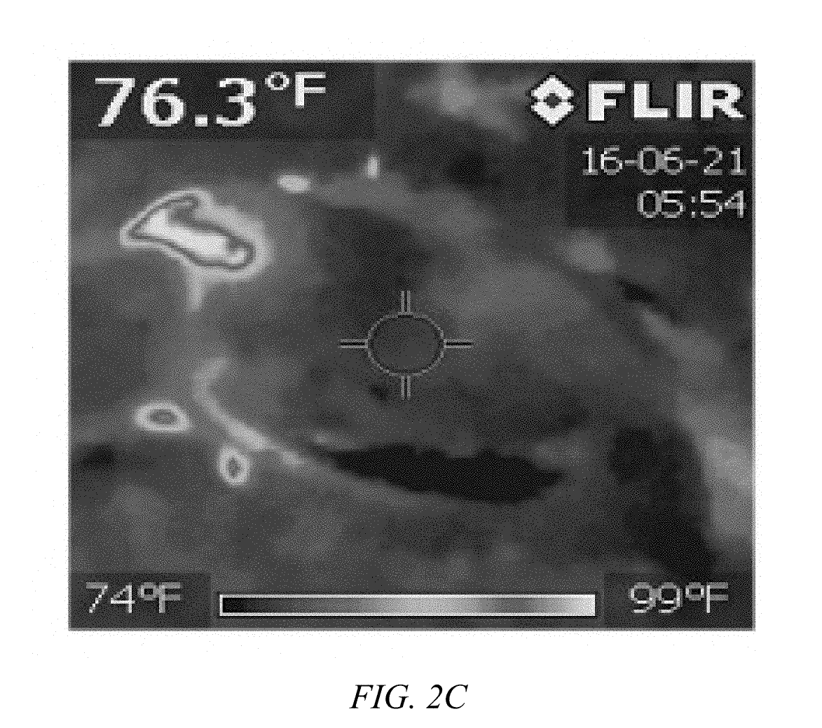

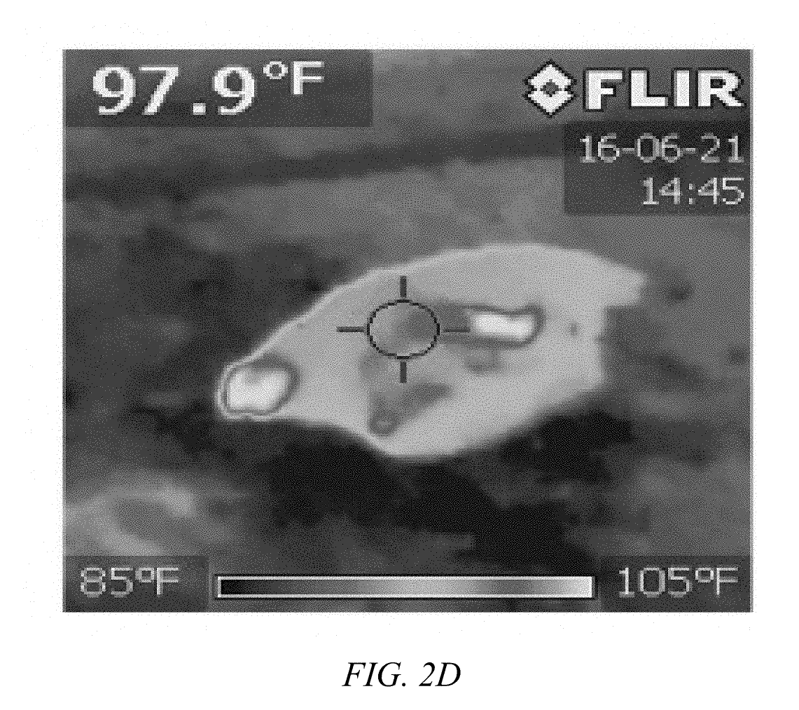

Five (5) week-old broilers were housed on floor pen under commercial condition during summer season (July 2016), and had ad libitum access to feed and clean water. Temperature of the barn were monitored continuously using HOBO U23 Pro v2 loggers (Onset Computer Corp., MA) and showed an average temperature of 24, and 29-30° C. in the morning (7 am), and afternoon (1 pm), respectively (FIG. 1). Core body temperatures (Tb) were recorded using Thermochron® temperature loggers (Maxim, CA) and showed a significant increase due to heat stress (HS) (FIGS. 2A and 2B). In line with Tb, skin and surface temperatures were also increased by HS as evaluated by thermal imaging camera (FIG. 2C). Growing feathers were collected at two time points (7 am and 1 pm) from the same bird. For a positive control, gut tissues (duodenum) were collected from two additional separate groups (thermoneutral, 24° C. and HS, 30° C.). All experimental procedures in this example were conducted in accordance with the recommendations in the guide for the care and use of laboratory animals of the National Institutes of Health and the protocol was reviewed and approved by the University of Arkansas Animal Care and Use Committee.

Feather Collection and Feather HSP70 Measurement:

Before collection, left breast area surrounding the growing feather was cleaned with ethanol 70% and growing feathers (n=3/bird) were gently removed in a sterile tube (1.5 mL), snap frozen in liquid nitrogen and stored at negative 80° C. until use.

Feathers were homogenized in lysis buffer (10 mM Tris-HCl (pH 8.0), 140 mM NaCl, 1.5 mM MgCl2, 0.5% Igepal, 2 mM vanadyl ribonuleoside complex) containing phosphatase and protease inhibitors using bullet blender (Nextadvance, NY). One-tenth of the lysate was added to 1 mL Trizol reagent (ThermoFisher Scientific, Waltham, Mass.) for total RNA isolation according to manufacturer's recommendations. The rest of the lysate was used for immunoblot as described below.

Total RNA (1 μg) was reverse transcribed using 4 μL qScript cDNA synthesis kit (Quanta Biosciences, Gaithersburg, Md.) in a total 20 μL reaction. The reverse transcription reaction was performed at 42° C. for 30 min followed by an incubation at 85° C. for 5 min. To determine the expression of HSP70 in the feather, real-time quantitative PCR (Applied Biosystems 7500 Real-Time PCR system) was performed using 5 μL of 10× diluted cDNA, 0.5 μM of each forward (5′-GGGAGAGGGTTGGGCTAGAG-3′) and reverse (5′-TTGCCTCCTGCCCAATCA-3′) specific primer for chicken HSP70, and SYBR Green Master Mix was used. Ribosomal 18S was used as a housekeeping gene. The qPCR cycling conditions were 50° C. for 2 min, 95° C. for 10 min followed by 40 cycles of a two-step amplification program (95° C. for 15 s and 58° C. for 1 min).

For HSP70 protein expression, total protein concentrations were determined using Synergy HT multi-mode microplate reader (BioTek, Winooski, Vt.) and a Bradford assay kit (Bio-Rad, Hercules, Calif.) with bovine serum albumin as a standard. Proteins (70 μg) were run on SDS page, transferred to PVDF membranes and blocked for 1 h at room temperature, and incubated with anti-HSP70 primary antibodies (diluted 1:1000) at 4° C. overnight. Pre-stained molecular weight marker (Precision Plus Protein Dual Color) was used as a standard (BioRad, Hercules, Calif.). The signal was visualized by enhanced chemiluminescence (ECL plus; GE Healthcare Bio-Sciences, Buckinghamshire, UK) and captured by FluorChem M MultiFluor System (Proteinsimple, Santa Clara, Calif.). Image Acquisition and Analysis were performed by AlphaView software (Version 3.4.0, 1993-2011, Proteinsimple, Santa Clara, Calif.).

Results: Feather HSP70 is Induced by HS

FIG. 1 graphically illustrates the ambient temperature in the barn, and as illustrated, data uploaded from HOBO loggers showed that the ambient temperature in the barn increased progressively during the day to reach 29-30° C. early in the afternoon compared to 23-24° C. in the morning (FIG. 1).

FIGS. 2A through 2D illustrate HS increased core body and surface temperature in broilers. As shown, this increased ambient temperature induced a significant increase in core body temperature (FIGS. 2A and 2B) as well as the skin and surface temperature of the chickens (FIGS. 2C and 2D). These data indicated that the chickens were under a stress due to heat load.

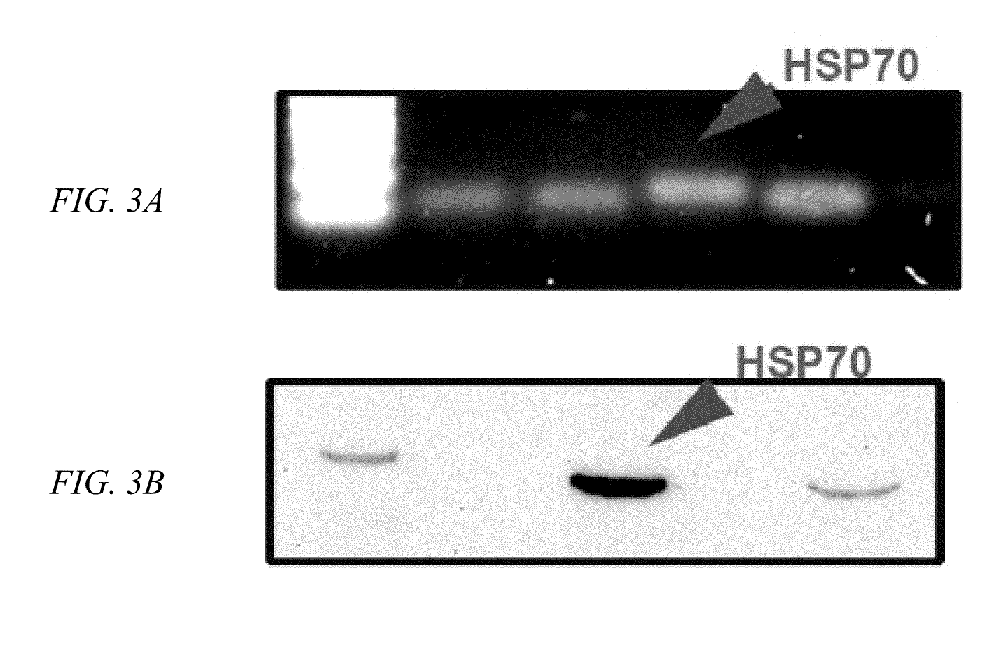

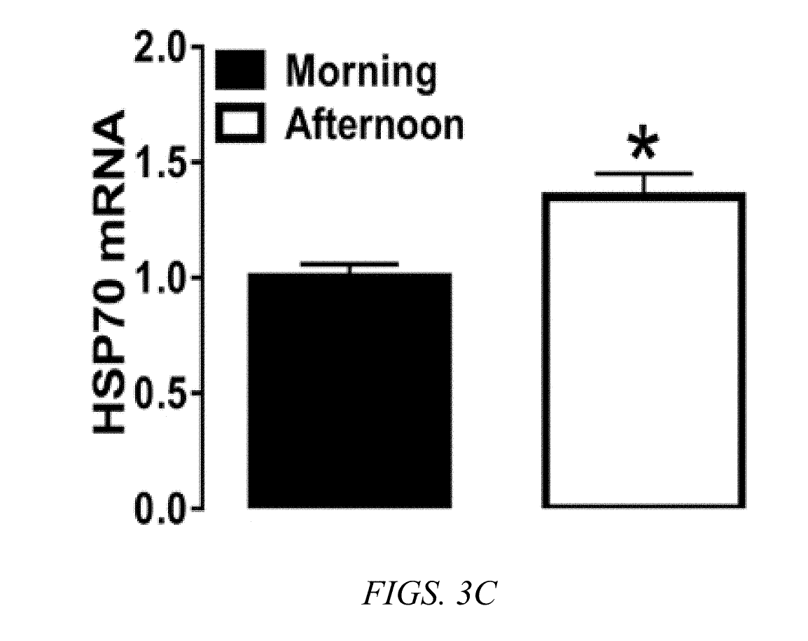

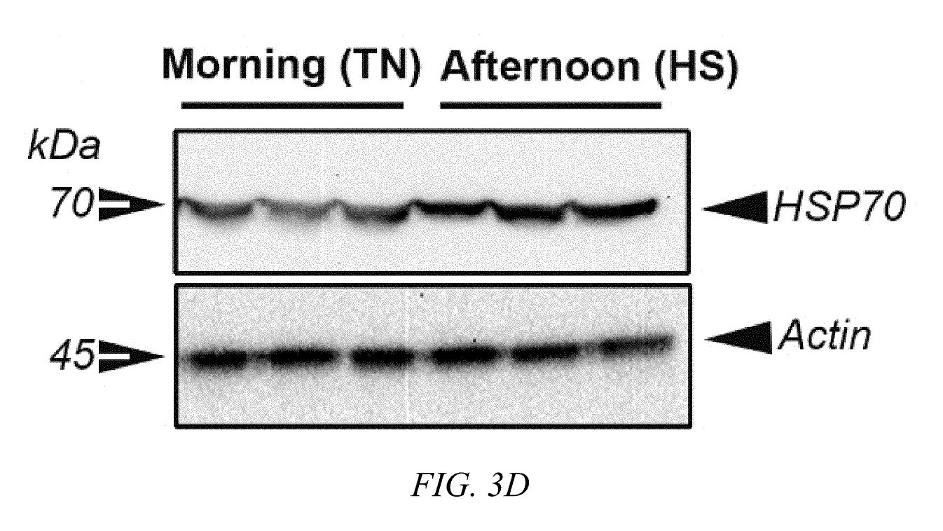

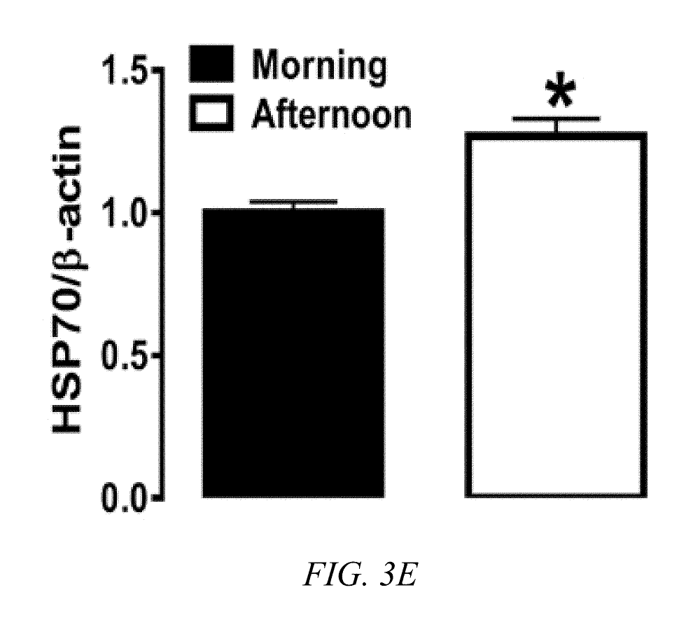

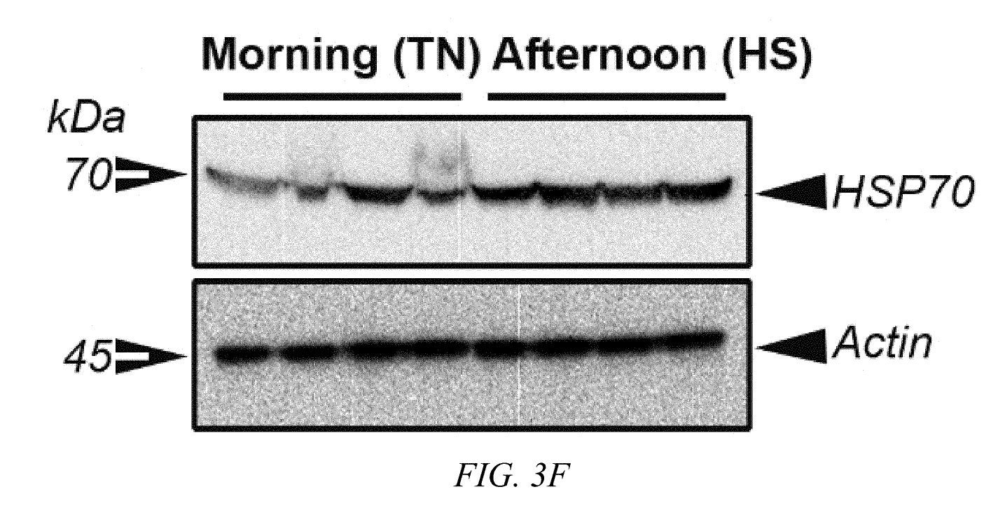

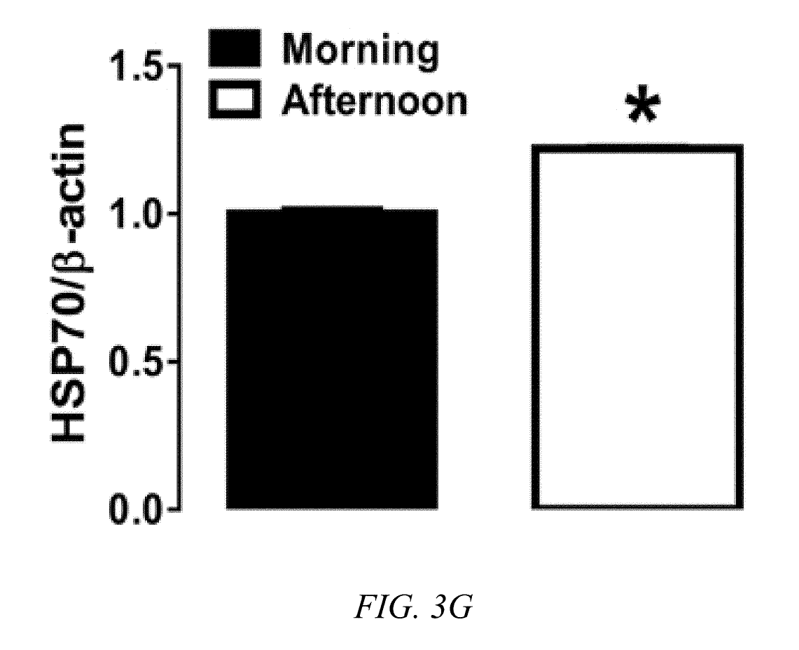

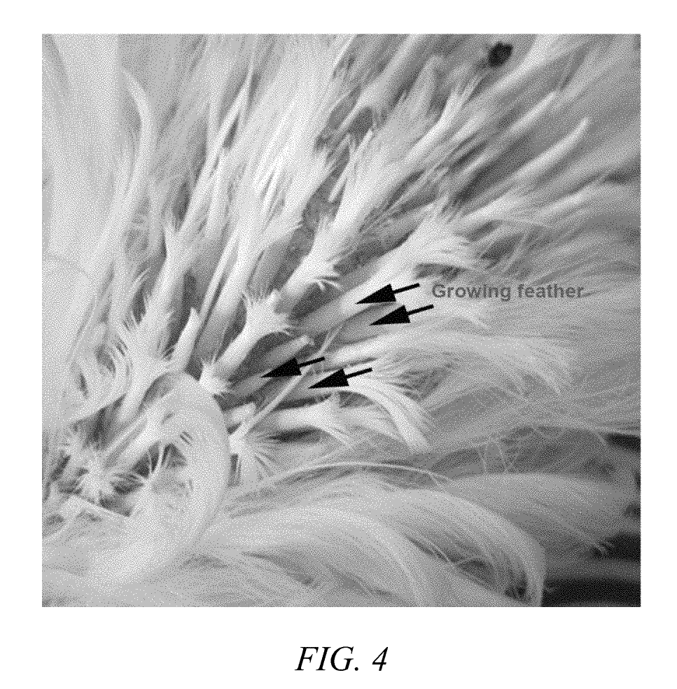

FIGS. 3A through 3G show that HSP70 mRNA and protein are highly expressed in chicken feather (FIGS. 3A and 3B) and are induced by heat stress (FIGS. 3C, 3D and 3E). As demonstrated herein, HSP70 (mRNA and protein) are highly expressed in chicken growing feathers (FIGS. 3A and 3B). As illustrated in FIG. 3C, heat stress increased HSP70 mRNA abundance in chicken feather compared to thermoneutral conditions (24° C. in the morning) (FIG. 3C). Similarly, feather HSP70 protein levels were concomitantly and significantly induced by heat stress (FIGS. 3D and E).

Since heat stress reduces intestinal barrier integrity, alter gut functions, induces leaky gut and increases intestinal HSP70 expression, gut (duodenum) was used as a positive control. As depicted in FIGS. 3F and 3G and similarly to the feather, heat stress increased HSP70 expression in the chicken gut with a similar magnitude as in the feather. Based on the experiment results provided herein, feather HSP70 is a reliable marker for stress induced by environmental heat in broilers. This marker is advantageous because it is non-invasive, rapid, and easy to use. In addition, feather tissues are easy accessible (without biopsy or euthanasia), and regenerate which allow a sequential study and stress monitoring in the same individual for long rearing period.

In addition to being a reliable marker for stress induced by environmental heat in broilers, feather HSP70 is also a reliable marker for other type of stress, such as transport, feed restriction, and in other poultry species (layers, turkeys, breeders, etc.).

It is to be understood that the terms “including”, “comprising”, “consisting” and grammatical variants thereof do not preclude the addition of one or more components, features, steps, or integers or groups thereof and that the terms are to be construed as specifying components, features, steps or integers.

If the specification or claims refer to “an additional” element, that does not preclude there being more than one of the additional element.

It is to be understood that where the claims or specification refer to “a” or “an” element, such reference is not be construed that there is only one of that element.

It is to be understood that where the specification states that a component, feature, structure, or characteristic “may”, “might”, “can” or “could” be included, that particular component, feature, structure, or characteristic is not required to be included.

Where applicable, although state diagrams, flow diagrams or both may be used to describe embodiments, the invention is not limited to those diagrams or to the corresponding descriptions. For example, flow need not move through each illustrated box or state, or in exactly the same order as illustrated and described.

Methods of the instant disclosure may be implemented by performing or completing manually, automatically, or a combination thereof, selected steps or tasks.

The term “method” may refer to manners, means, techniques and procedures for accomplishing a given task including, but not limited to, those manners, means, techniques and procedures either known to, or readily developed from known manners, means, techniques and procedures by practitioners of the art to which the invention belongs.

For purposes of the instant disclosure, the term “at least” followed by a number is used herein to denote the start of a range beginning with that number (which may be a ranger having an upper limit or no upper limit, depending on the variable being defined). For example, “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. Terms of approximation (e.g., “about”, “substantially”, “approximately”, etc.) should be interpreted according to their ordinary and customary meanings as used in the associated art unless indicated otherwise. Absent a specific definition and absent ordinary and customary usage in the associated art, such terms should be interpreted to be ±10% of the base value.

When, in this document, a range is given as “(a first number) to (a second number)” or “(a first number)-(a second number)”, this means a range whose lower limit is the first number and whose upper limit is the second number. For example, 25 to 100 should be interpreted to mean a range whose lower limit is 25 and whose upper limit is 100. Additionally, it should be noted that where a range is given, every possible subrange or interval within that range is also specifically intended unless the context indicates to the contrary. For example, if the specification indicates a range of 25 to 100 such range is also intended to include subranges such as 26-100, 27-100, etc., 25-99, 25-98, etc., as well as any other possible combination of lower and upper values within the stated range, e.g., 33-47, 60-97, 41-45, 28-96, etc. Note that integer range values have been used in this paragraph for purposes of illustration only and decimal and fractional values (e.g., 46.7-91.3) should also be understood to be intended as possible subrange endpoints unless specifically excluded.

It should be noted that where reference is made herein to a method comprising two or more defined steps, the defined steps can be carried out in any order or simultaneously (except where context excludes that possibility), and the method can also include one or more other steps which are carried out before any of the defined steps, between two of the defined steps, or after all of the defined steps (except where context excludes that possibility).

Still further, additional aspects of the invention may be found in one or more appendices attached hereto and/or filed herewith, the disclosures of which are incorporated herein by reference as if fully set out at this point.

Thus, the invention is well adapted to carry out the objects and attain the ends and advantages mentioned above as well as those inherent therein. While the inventive concept has been described and illustrated herein by reference to certain illustrative embodiments in relation to the drawings attached thereto, various changes and further modifications, apart from those shown or suggested herein, may be made therein by those of ordinary skill in the art, without departing from the spirit of the inventive concept the scope of which is to be determined by the following claims.

What is claimed is:

1. A non-invasive method of detecting stress in a poultry species, said method comprising the steps of:
(a) obtaining a growing feather sample from said poultry species; and
(b) detecting the expression of HSP70 in said growing feather sample, wherein expression of HSP70 is indicative of said stress in said poultry species.

2. The method of claim 1 further comprising the step of:
assaying said growing feather sample for the expression level of at least one stress-related marker, said marker being a protein encoded by the gene of HSP70 or fragments of said protein which are immunoreactive to HSP70.

3. The method of claim 1 further comprising the step of determining the amount of HSP70 in the growing feather sample, wherein an elevated amount of HSP70 indicates the presence of stress in the poultry species.

4. The method of claim 1 further comprising the step of:
(c) diagnosing said poultry species with said stress when HSP70 is detected in said sample isolated from said feather sample.

5. The method of claim 1 wherein said stress is stress induced by environmental heat or cold, feed restrictions, transport or a combination thereof.

6. The method of claim 1 wherein said poultry species is selected from the group consisting of chickens, turkeys, or quail.

7. The method of claim 6 wherein said poultry species is broiler chickens.

8. The method of claim 1 wherein said sample is non-invasively obtained without biopsy or euthanasia.

9. A polymerase chain reaction (PCR) kit for use with the method of claim 1.

10. An enzyme-linked immunosorbent assay (ELISA) kit for use with the method of claim 1.

11. A method of detecting stress in a poultry species, said method comprising the steps of:
(a) non-invasively obtaining a sample of at least one growing feather from said poultry species; and
(b) assaying said growing feather sample for the expression level of at least one stress-related marker, said marker being a protein encoded by the gene of HSP70 or fragments of said protein which are immunoreacitve to HSP70, wherein expression of HSP70 is indicative of said stress in said poultry species.

12. The method of claim 11 wherein said stress is stress induced by environmental heat or cold, feed restrictions, transport or a combination thereof.

13. The method of claim 11 wherein said poultry species is selected from the group consisting of chickens, turkeys, or quail.

14. The method of claim 13 wherein said poultry species is broiler chickens.

15. The method of claim 11 wherein said growing feather sample is non-invasively obtained without biopsy or euthanasia.

16. A polymerase chain reaction (PCR) kit for use with the method of claim 11.

17. An enzyme-linked immunosorbent assay (ELISA) kit for use with the method of claim 11.

18. A method of determining stress induced by stress in a living poultry subject, said method comprising the steps of:
(a) obtaining a biological sample of at least one growing feather from said living poultry subject;
(b) extracting or otherwise isolating genomic DNA from said biological sample;
(c) treating said genomic DNA, or a fragment thereof, with one or more reagents specific for HSP70 as a stress-related marker;
(d) detecting the expression HSP70; and
(c) determining said stress in said living poultry subject when the presence of HSP70 from said biological sample is detected.

19. The method of claim 18 further comprising the step of determining the amount of HSP70 in the biological sample, wherein an elevated amount of HSP70 indicates the presence of stress in the living poultry subject.

20. The method of claim 18 wherein said stress is stress induced by environmental heat or cold, feed restrictions, transport or a combination thereof.

21. The method of claim 18 wherein said living poultry subject is selected from the group consisting of a chicken, a turkey, or a quail.

22. The method of claim 21 wherein said living poultry subject is a broiler chicken.

23. A polymerase chain reaction (PCR) kit for use with the method of claim 18.

24. An enzyme-linked immunosorbent assay (ELISA) kit for use with the method of claim 18.
